(19) United States
(12) Patent Application Publication
(10) Pub. No.: US 2025/0064997 A1
(43) Pub. Date: Feb. 27, 2025

(54) USING SILVER COMPOSITIONS TO DISINFECT SURFACES

(71) Applicant: SilverCeuticals Inc., Lindon, UT (US)

(72) Inventors: Keith W. Moeller, Highland, UT (US); Cameron D. Moeller, Pleasant Grove, UT (US); Kirk W. Moeller, American Fork, UT (US); Kory R. Dopp, Eagle Mountain, UT (US); Bracken J. Moeller, Lehi, UT (US); Kimball K. Moeller, American Fork, UT (US); Brennan R. Moeller, Highland, UT (US)

(21) Appl. No.: 18/726,344

(22) PCT Filed: Nov. 3, 2022
(86) PCT No.: PCT/US2022/079208

§ 371 Date: Jul. 2, 2024

(51) Int. Cl. A61L 2/00; A01N 25/04; A01N 59/00; A01N 59/16; A01P 1/00; A61L 2/18; A61L 2/238; A61L 2/24; A61L 101/02

(52) U.S. Cl. CPC A61L 2/0088; A01N 25/04; A01N 59/00; A01N 59/16; A01P 1/00; A61L 2/186; A61L 2/238; A61L 2/24; A61L 2101/02; A61L 2202/14; A61L 2202/15

(57) ABSTRACT

Methods, apparatus, and computer program products for using a silver composition to disinfect surfaces are disclosed. One method (400, 900) includes providing (402, 902) a composition including H₂O₂ and silver particles (100) to an applicator device (300, 600) and applying (404, 904) the composition to a target type of surface. An apparatus includes an applicator device (600) configured to store a composition including H₂O₂ and silver particles (100), a processor (608), and a memory (606) to automatedly apply the composition to a target type of surface. One computer program product includes a computer-readable storage device (606) including code embodied therewith for using silver compositions to disinfect surfaces. The code is executable by a processor (608) to automatedly facilitate providing a composition including H₂O₂ and silver particles (100) to an applicator device (600) and automatedly facilitate applying the composition to a target type of surface.

### FIELD

The subject matter disclosed herein relates to silver compositions and more particularly relates to methods, apparatus, and program products for using silver compositions to disinfect surfaces.

### BACKGROUND

Many environments are exposed to and can include microbes (e.g., bacteria, viruses, fungi, archaea, and protists, etc.). Some microbes are capable of causing a variety of diseases and are referred to as, pathogens. Pathogens can be found on a variety of surfaces within an environment. That is, because one or more surfaces within an environment can harbor one or more pathogens, at least some environments can facilitate the spread of disease.

While many compositions are capable of disinfecting a surface by destroying the pathogen(s) that may be found thereon, these compositions include one or more drawbacks. Some drawbacks of conventional compositions that are used to disinfect a surface include, for example, a lack of overall efficacy to disinfect a surface, a short and/or relatively short efficacy period of time, a reduced efficacy period of time, being toxic to humans and/or animals, being harmful to the environment, being harmful to the treated surface (e.g., staining, corrosive, etc.), and/or being dangerous/hazardous to handle (e.g., flammable, volatile, etc.), among other draw backs that are possible.

### BRIEF SUMMARY

The subject matter of the present disclosure provides examples of silver compositions and corresponding methods, apparatus, and computer program products that overcome the above-discussed shortcomings of prior art compositions and/or techniques. There is a desire to utilize silver compositions to disinfect a variety of surfaces and/or types of surfaces to prevent or at least reduce the spread of disease caused by the pathogen(s) that may be harbored on the surfaces and/or types of surfaces. It would therefore be desirable to develop improved methods, apparatus, and computer program products for using silver compositions to disinfect a variety of surfaces and/or types of surfaces. Accordingly, the subject matter of the present application has been developed in response to the present state of the art, and in particular, in response to the shortcomings of conventional disinfectants and/or the shortcomings of conventional methods, apparatus (and/or systems), and computer program products for disinfecting surfaces.

Disclosed herein is a method for using silver compositions to disinfect surfaces. The method includes providing a composition comprising hydrogen peroxide (H₂O₂) and silver (Ag) particles to an applicator device and applying the composition to a target type of surface. The preceding subject matter of this paragraph characterizes example 1 of the present disclosure.

The composition comprises 0.1% to 5% H₂O₂. The preceding subject matter of this paragraph characterizes example 2 of the present disclosure, wherein example 2 also includes the subject matter according to example 1, above.

The composition comprises 10 ppb to 60 ppm of the Ag particles. The preceding subject matter of this paragraph characterizes example 3 of the present disclosure, wherein example 3 also includes the subject matter according to any one of examples 1 or 2, above.

The composition comprises 3% H₂O₂ and 10 ppm of the Ag particles. The preceding subject matter of this paragraph characterizes example 4 of the present disclosure, wherein example 4 also includes the subject matter according to example 1, above.

The Ag particles comprise one of nano-silver particles, colloidal silver particles, and ionic silver particles. The preceding subject matter of this paragraph characterizes example 5 of the present disclosure, wherein example 5 also includes the subject matter according to any one of examples 1 or 4, above.

The applicator device comprises a manually operated applicator device, the manually operated applicator device comprises one of a manually operated sprayer, a manually operated fogger, a manually operated mister, a manually operated sprinkler, a manually operated spout, a manually operated mop, a manually operated wiper, a manually operated diffuser, a manually operated nebulizer, and a manually operated soaker, and applying the composition comprises manually applying the composition via the manually operated applicator device. The preceding subject matter of this paragraph characterizes example 6 of the present disclosure, wherein example 6 also includes the subject matter according to example 1, above.

The applicator device includes an automated applicator device, the automated applicator device comprises one of an automated sprayer, an automated fogger, an automated mister, an automated sprinkler, an automated spout, an automated mop, an automated wiper, an automated diffuser, an automated nebulizer, and an automated soaker, and applying the composition comprises automatedly applying the composition via the automated applicator device. The preceding subject matter of this paragraph characterizes example 7 of the present disclosure, wherein example 7 also includes the subject matter according to example 1, above.

The target type of surface comprises one of a hard surface, a firm surface, a soft surface, and an agricultural surface. The preceding subject matter of this paragraph characterizes example 8 of the present disclosure, wherein example 8 also includes the subject matter according to example 1, above.

Applying the composition to the target type of surface comprises applying the composition to the one of the hard surface, the firm surface, the soft surface, and the agricultural surface at a first time and at one or more second times that are not less than twenty-one days after a previous application of the composition to the one of the hard surface, the firm surface, and the agricultural surface. The preceding subject matter of this paragraph characterizes example 9 of the present disclosure, wherein example 9 also includes the subject matter according to any one of examples 1 or 8, above.

Applying the composition to the target type of surface comprises applying the composition to the target type of surface at a first time and at one or more second times that are not less than twenty-one days after a previous application of the composition to the target type of surface. The preceding subject matter of this paragraph characterizes example 10 of the present disclosure, wherein example 10 also includes the subject matter according to example 1, above.

Further disclosed herein is an apparatus for using silver compositions to disinfect surfaces. The apparatus comprises an applicator device configured to store a composition comprising H₂O₂ and Ag particles, a processor, and a memory configured to store code executable by the processor to automatedly apply, via the applicator device, the composition to a target type of surface. The preceding subject matter of this paragraph characterizes example 11 of the present disclosure.

The composition comprises 0.1% to 5% H₂O₂. The preceding subject matter of this paragraph characterizes example 12 of the present disclosure, wherein example 12 also includes the subject matter according to example 11, above.

The composition comprises 10 ppb to 60 ppm of the Ag particles. The preceding subject matter of this paragraph characterizes example 13 of the present disclosure, wherein example 13 also includes the subject matter according to any one of examples 11 or 12, above.

The composition comprises 3% H₂O₂ and 10 ppm of the Ag particles. The preceding subject matter of this paragraph characterizes example 14 of the present disclosure, wherein example 14 also includes the subject matter according to example 11, above.

The Ag particles comprise one of nano-silver particles, colloidal silver particles, and ionic silver particles. The preceding subject matter of this paragraph characterizes example 15 of the present disclosure, wherein example 15 also includes the subject matter according to any one of examples 11 or 14, above.

The applicator device includes an automated applicator device, the automated applicator device comprises one of an automated sprayer, an automated fogger, an automated mister, an automated sprinkler, an automated spout, an automated mop, an automated wiper, an automated diffuser, an automated nebulizer, and an automated soaker, and automatedly applying the composition comprises automatedly applying the composition via the automated applicator device. The preceding subject matter of this paragraph characterizes example 16 of the present disclosure, wherein example 16 also includes the subject matter according to example 11, above.

The target type of surface comprises one of a hard surface, a firm surface, a soft surface, and an agricultural surface. The preceding subject matter of this paragraph characterizes example 17 of the present disclosure, wherein example 17 also includes the subject matter according to example 11, above.

Automatedly applying the composition to the target type of surface comprises automatedly applying the composition to the one of the hard surface, the firm surface, the soft surface, and the agricultural surface at a first time and at one or more second times that are not less than twenty-one days after a previous application of the composition to the one of the hard surface, the firm surface, the soft surface, and the agricultural surface. The preceding subject matter of this paragraph characterizes example 18 of the present disclosure, wherein example 18 also includes the subject matter according to any one of examples 11 or 17, above.

Applying the composition to the target type of surface comprises applying the composition to the target type of surface at a first time and at one or more second times that are not less than twenty-one days after a previous application of the composition to the target type of surface. The preceding subject matter of this paragraph characterizes example 19 of the present disclosure, wherein example 19 also includes the subject matter according to example 11, above.

Additionally, disclosed herein is a computer program product comprising a computer-readable storage device including code embodied therewith for using silver compositions to disinfect surfaces. The code is executable by a processor to cause the processor to automatedly facilitate providing a composition comprising hydrogen peroxide and silver particles to an applicator device and automatedly facilitate applying, via the applicator device, the composition to a target type of surface. The preceding subject matter of this paragraph characterizes example 20 of the present disclosure.

### BRIEF DESCRIPTION OF THE DRAWINGS

A more particular description of the embodiments briefly described above will be rendered by reference to specific embodiments that are illustrated in the appended drawings. Understanding that these drawings depict only some embodiments and are not therefore to be considered to be limiting of scope, the embodiments will be described and explained with additional specificity and detail through the use of the accompanying drawings, in which:

FIG. 1 is a schematic diagram of one embodiment of a silver particle;

FIG. 2 is a schematic diagram of one embodiment of a molecule of coating for the silver particle illustrated in FIG. 1;

FIG. 3 is a schematic block diagram of one embodiment of a manually operated applicator device for manually applying a silver composition to a target surface and/or target type of surface;

FIGS. 4 and 5 are schematic flow diagrams of various embodiments of a method for using a silver composition to manually disinfect a target surface and/or target type of surface;

FIGS. 6A and 6B are schematic block diagrams of various embodiments of an automatedly and/or automatically operated applicator device for automatedly and/or automatically applying a silver composition to a target surface and/or target type of surface;

FIGS. 7A and 7B are schematic block diagrams of various embodiments of a memory device included in the applicator devices of FIGS. 6A and 6B;

FIGS. 8A and 8B are schematic block diagrams of various embodiments of a processor included in the applicator devices of FIGS. 6A and 6B; and

FIGS. 9 and 10 are schematic flow diagrams of various embodiments of a method for using a silver composition to automatedly and/or automatically disinfect a target surface and/or target type of surface.

### DETAILED DESCRIPTION

The various embodiments disclosed herein provide silver compositions and methods that can disinfect a variety of surfaces. The silver compositions include a combination of silver particles and hydrogen peroxide that, when applied to a surface, can disinfect that surface.

As disclosed herein, “silver particles” can include, but are not limited to, “nano-silver particles,” “nano-silver,” “colloidal silver particles,” “colloidal silver,” “ionic silver particles,” “ionic silver,” “solution silver particles,” “solution silver,” “suspension silver particles,” and “suspension silver,” among other particles that can include silver that are possible and contemplated herein. In some embodiments, “silver particles” refers to particles that, in whole or in part, comprise silver in an aqueous medium or solution.

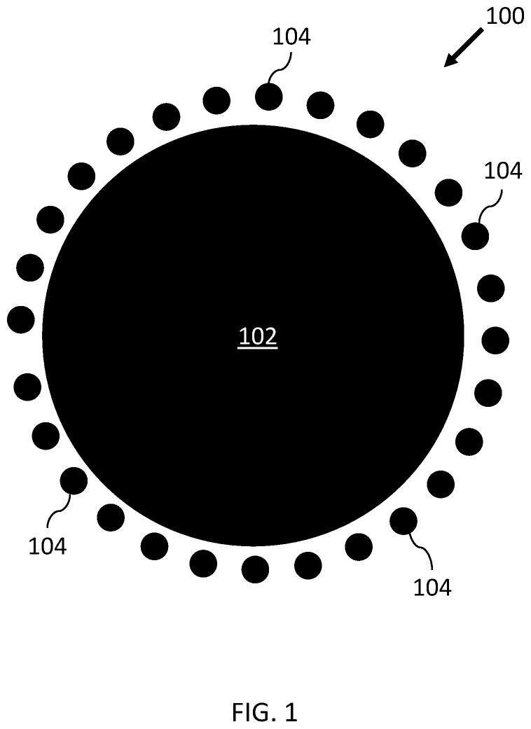

With reference to the drawings, FIG. 1 is a diagram of one embodiment of a silver particle 100, which can also be referred to herein as, an Ag particle. A silver particle 100 may be characterized in terms of valence of the silver included therein. In the exemplary embodiment shown in FIG. 1, the silver particle 100 includes a silver core 102 and a silver coating 104.

A silver core 102, in various embodiments, includes metallic silver (Ag (0)) to form a metallic silver core. In additional embodiments, the silver core 102 includes metallic nano-silver to form a metallic nano-silver core.

In various embodiments, the silver coating 104 comprises Ag(I), Ag(II), or Ag(III) in an ionic oxidation state. In some embodiments, the silver coating 104 includes silver oxide (e.g., AgO). That is, the silver particle 100 comprises Ag(0) and AgO, where AgO is present as a coating on the silver core 102 (e.g., a metallic nano-silver core).

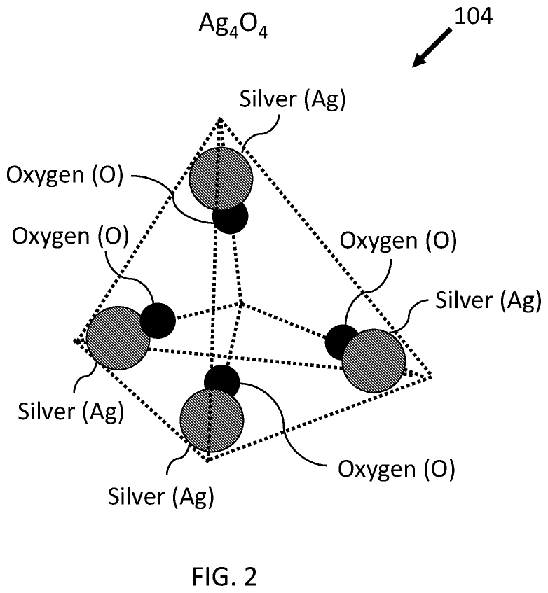

In other embodiments (see, FIG. 2), the silver coating 104 may be at least partially in the form of silver II oxide (Ag₄O₄). In a molecule of this material, two of the silver atoms are in the 1+ state (silver I) and the other two silver molecules are in the 3+ state (silver III). Under certain conditions, these molecules can give rise to silver atoms (Ag) in the 2+ (silver II) state attached to respective oxygen atoms (O). Thus, the present disclosure provides silver particles comprising a metallic silver core 102 and silver coating 104 of silver oxide, the silver oxide being selected from AgO and Ag₄O₄.

The purity of a silver particle 100 may be any suitable percentage of silver purity that is capable of disinfecting a surface. In various embodiments, the percentage of silver purity of a silver particle 100 is in the range of about 50% to about 99.9999%, among other percentages of silver purity that are greater than 99.9999% or less than 50% that are possible and contemplated herein. In certain embodiments, the percentage of silver purity of a silver particle 100 is about 99.99%, among other percentages of silver purity that are greater than or less than about 99.99% that are possible and contemplated herein.

In various embodiments, a plurality of silver particles 100 are added to and/or mixed with one or more liquids and/or solutions to form a silver composition that can be used to disinfect a variety of surfaces. The silver particles 100 can be added to and/or mixed with any suitable liquid that can facilitate, enable, and/or allow the silver particles 100 to disinfect a surface.

In various embodiments, one liquid includes water (H₂O) or other aqueous solution. In some embodiments, the water or aqueous solution includes pure water and/or purified water.

An aqueous composition comprising silver particles 100 may be prepared using any suitable method and/or technique that is known or developed in the future. For example, the preparation of a composition comprising silver particles 100 may utilize an electrochemical cell comprising electrodes. One process includes: (a) placing a silver electrode in contact with a quantity of high purity water; (b) conveying electrical current through the silver electrode to thereby separate particles of silver from the silver electrode in a manner sufficient to cause production of suspended silver particles within the water; and (c) agitating the water during said production of suspended silver particles to thereby disperse the silver particles into a more uniform concentration within the water such that a higher quantity of silver particles can be produced per batch.

Another example technique for preparing a composition comprising silver particles 100 includes: (a) establishing an electrical circuit comprising a current source, and a first conductor electrically connected to the current source and a second conductor electrically connected to the current source in which the first conductor is spaced apart from the second conductor, and at least one of the conductors is made of elemental silver; (b) closing the circuit by placing the first conductor and the second conductor in communication with a fluidic resistor; (c) operating the current source to supply alternating current simultaneously to the first conductor and the second conductor such that voltage is increasing and decreasing within the first and second conductors in alternating tandem to thereby cause silver particles to separate from the first electrode and enter the fluidic resistor and become disposed in suspension within the fluidic resistor; and (d) selectively adjusting the electrodes by moving them toward the fluidic resistor to compensate for decrease in electrode length due to gradual separation of silver particles therefrom to thereby prevent arcing from occurring between the electrodes and said fluidic resistor.

In various embodiments, the total amount of silver particles 100 in a silver composition that can disinfect a surface is in the range of about ten parts per billion (10 ppb) to about sixty parts per million (60 ppm), among other quantities of silver particles 100 that are less than 10 ppb and greater than 60 ppm that are possible and contemplated herein. In some embodiments, the total amount of silver particles in a composition is about 10 ppm, among other amounts of silver particles 100 greater than or less that about 10 ppm that are possible and contemplated herein. In other embodiments, the total amount of silver particles in a composition is about 32 ppm, among other amounts of silver particles 100 greater than or less that about 32 ppm that are possible and contemplated herein.

As the silver particles 100 become smaller, a given concentration of silver particles 100 will represent a larger quantity of particles 100. In addition, the total surface area for a given particle concentration will increase. Therefore, particle size and/or range of particle size for the silver particles 100 may further characterize the compositions of the various embodiments, which may include any suitable particle size and/or range of particle size that is/are known or developed in the future capable of disinfecting surfaces.

In some embodiments, a silver composition comprises 50-100% of the silver particles 100 including a maximum dimension that is less than about 1.0 micrometer (1000 nanometers (nm)), among other percentages and sizes that are possible and contemplated herein. In certain embodiments, a silver composition comprises 50-100% of the silver particles 100 including a dimension in the range of about 0.005 micrometers (5 nm) to about 0.01 micrometers (10 nm), among other percentages and sizes that are possible and contemplated herein. In still other embodiments, a silver composition comprises 50-100% of the silver particles 100 including a minimum dimension greater than or equal to about 0.001 micrometers (1 nm), among other percentages and sizes that are possible and contemplated herein. Here, these silver particles 100 can be considered colloidal silver particles.

In further embodiments, a silver composition comprises 50-100% of the silver particles 100 including a maximum dimension that is less than about 0.001 micrometers (1 nm), among other percentages and sizes that are possible and contemplated herein. Here, these silver particles 100 can be considered ionic silver particles or solution silver particles.

In still further embodiments, a silver composition comprises 50-100% of the silver particles 100 including a minimum dimension that is greater than about 1.0 micrometer (1000 nm), among other percentages and sizes that are possible and contemplated herein. Here, these silver particles 100 can be considered suspension silver particles.

In one embodiment, the silver particles 100 are stable in essentially and/or substantially pure water that is free of, for example, surfactants, etc., among other natural impurities and/or unnatural impurities that are possible and contemplated herein. Additionally, or in another embodiment, the composition of silver particles 100 is essentially and/or substantially colorless, which can prevent and/or substantially prevent discoloring (e.g., staining, bleaching, etc.) one or more target surfaces and/or one or more target types of surfaces or at least facilitate preventing and/or substantially preventing discoloring one or more target surfaces and/or one or more target types of surfaces when the composition of silver particles 100 is applied to and/or otherwise comes into contact with the target surface(s) and/or target type(s) of surface(s).

In various embodiments, the silver composition further comprises an amount of hydrogen peroxide (H₂O₂). That is, the silver composition includes at least a plurality of silver particles 100 and a predetermined amount of H₂O₂.

The silver composition may include any suitable amount of H₂O₂ that, in combination with the silver particles 100, can disinfect a variety of surfaces. In various embodiments, the silver composition includes an amount of H₂O₂ in the range of about 0.1% to about 10% H₂O₂, among other ranges and/or percentages of H₂O₂ that are greater than about 10% or less than about 0.1% H₂O₂ that are possible and contemplated herein. In certain embodiments, the silver composition includes about 3% H₂O₂, among other percentages of H₂O₂ that are greater than or less than about 3% H₂O₂ that are possible and contemplated herein.

In various embodiments, a silver composition includes silver particles 100, a predetermined amount and/or percentage of H₂O₂ (e.g., by volume), and water (e.g., pure and/or purified water). These compositions may be prepared by combining the silver particles 100, H₂O₂, and water and thoroughly mixing the combination to provide a homogeneous composition. The amount or concentration of the silver particles 100 and/or H₂O₂ in the composition should be an amount that is suitable for disinfecting one or more target surfaces and/or one or more target types of surfaces.

In various embodiments, the silver composition forms a silver sol. A silver sol, in various embodiments, includes a stable dispersion of solid silver particles 100 homogeneously dispersed in a liquid. In certain embodiments, the silver sol includes a stable dispersion of solid silver particles 100 homogeneously dispersed in water (e.g., pure) and/or an aqueous solution. In further embodiments, the silver sol includes a stable dispersion of solid silver particles 100 homogeneously dispersed in a liquid that includes an amount of H₂O₂ (e.g., about 0% to about 100% H₂O₂, inclusive [0%-100%]). In still further embodiments, the silver sol includes a stable dispersion of solid silver particles 100 homogeneously dispersed in a liquid that includes an amount of H₂O₂ and water (e.g., pure) and/or an aqueous solution.

Testing of various embodiments of the silver composition (e.g., silver compositions including the silver particle(s) 100 shown in FIG. 1 (and FIG. 2)) that is free of H₂O₂ and/or does not include H₂O₂ (e.g., a hydrogen peroxide-free silver composition) shows that the hydrogen peroxide-free silver composition includes a pathogen kill rate in the range of about 99.9% to about 99.99%, depending on the logs of bacteria tested. However, the hydrogen peroxide-free silver composition is comparatively slow to achieve the initial kill and can take some time to achieve those kill rate results (e.g., 15 minutes to 4 hours for a total kill time on 6-logs of certain bacteria). The testing further shows that the hydrogen peroxide-free silver composition has a comparatively long-lasting kill rate that can keep killing pathogens for several weeks after the initial application (e.g., 21 days or longer).

Testing of various embodiments of H₂O₂ compositions that are free of silver have a relatively fast initial kill rate, with a complete kill of all pathogens tested (e.g., less than 5 minutes on all pathogens tested). The testing further shows that this pathogen kill rate for the H₂O₂ composition has little to no lasting efficacy and is no longer antimicrobial within a few minutes after its initial application, which is similar to combinations of H₂O₂ and other silver compositions (e.g., silver particles other than the silver particle 100 shown in FIG. 1 (and FIG. 2)).

In addition, testing of various embodiments of the silver composition that includes H₂O₂ (e.g., a silver-hydrogen peroxide silver composition) shows that the silver-hydrogen peroxide silver composition includes a pathogen kill rate in the range of about 99.9% to about 99.9999%, depending on the logs of bacteria tested. The silver-hydrogen peroxide silver composition has a relatively fast initial kill, with a complete kill of all pathogens tested (e.g., in less than 5 minutes on all pathogens tested). The testing further shows that this pathogen kill rate for the silver-hydrogen peroxide silver composition continues to have a relatively long-lasting kill rate that can keep killing pathogens for many weeks after the initial application (e.g., 21 days or longer).

Further testing shows that the pathogen kill rate for the silver-hydrogen peroxide silver composition on various target surfaces and/or various target types of surfaces can last up to about three (3) weeks or about twenty-one (21) days longer than other hydrogen peroxide-free silver compositions (e.g., silver compositions including silver particles other than the silver particle 100 shown in FIG. 1 (and FIG. 2)). That is, the combination of silver particles 100 and H₂O₂ provides the unexpected benefit of a faster initial kill and lengthening the efficacy of the pathogen kill rate compared to the use of other silver particles (e.g., silver particles other than the silver particle 100 shown in FIG. 1 (and FIG. 2)), silver particles 100 alone, or H₂O₂ alone. Accordingly, at least one embodiment of the silver-hydrogen peroxide silver composition includes a 99.9999% pathogen kill rate in less than about 5 minutes with an efficacy period of at least 21 days.

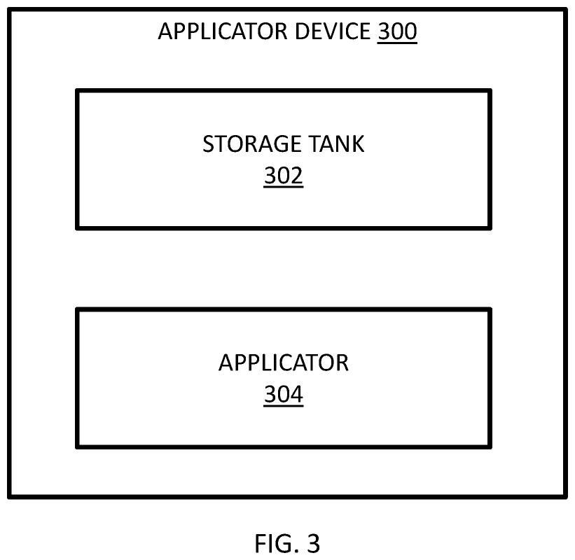

Referring to FIG. 3, FIG. 3 is a block diagram of one embodiment of an applicator device 300 for disinfecting surfaces. The applicator device 300 may include any suitable system, apparatus, and/or device that is known or developed in the future capable of manually applying a silver composition to one or more target surfaces and/or one or more target types of surfaces and/or otherwise facilitating contact of the silver composition and the target surface(s) and/or target type(s) of surface(s).

In various embodiments, the applicator device 300 includes a manually operated applicator device 300. Examples of a manually operated applicator device 300 can include, but are not limited to, a manually operated sprayer, a manually operated fogger, a manually operated mister, a manually operated sprinkler, a manually operated spout, a manually operated mop, a manually operated wiper, a manually operated diffuser, a manually operated nebulizer, and/or a manually operated soaker, etc., among other manually operated applicators that can apply a silver composition to one or more target surfaces and/or one or more target types of surfaces and/or otherwise facilitating contact of the silver composition and the target surface(s) and/or target type(s) of surface(s).

The applicator device 300 can apply a silver composition to the target surface(s) and/or target type(s) of surface(s) so that the silver composition can kill and/or destroy, on contact, one or more of the pathogens that may be present and/or harbored on the target surface(s) and/or target type(s) of surface(s). In various embodiments, the applicator device 300 can be used to apply a silver composition at regular and/or substantially regular intervals of time to maintain a pathogen-free surface and/or type of surface. In some embodiments, the regular and/or substantially regular interval of time can include application of the silver composition every 3 weeks or 21 days, among other intervals of time that are greater than or less than 3 weeks that are possible and contemplated herein.

At least in the embodiment illustrated in FIG. 3, the applicator device 300 includes, among other components, a storage tank 302 and an applicator 304 in fluid communication with the storage tank 302. A storage tank 302 may include any suitable tank and/or type of tank that is known or developed in the future capable of storing a composition, sol, liquid, solution, fluid, and/or liquid including a plurality of silver particles 100 or a mixture/combination of silver particles 100 and H₂O₂. Further, the storage tank 302 may include any suitable size and/or dimensions for storing/housing a predetermined and/or predefined amount of the composition, sol, liquid, solution, fluid, and/or liquid including a plurality of silver particles 100 or a mixture/combination of silver particles 100 and H₂O₂.

An applicator 304 may include any suitable applicator and/or type of applicator that is known or developed in the future that is used manually. That is, an applicator 304 may include any suitable device and/or apparatus that can apply, via manual operation, a composition, a sol, liquid, solution, fluid, and/or liquid including a plurality of silver particles 100 or a mixture/combination of silver particles 100 and H₂O₂ to one or more target surfaces and/or one or more target types of surfaces and/or otherwise facilitating contact of the silver composition and the target surface(s) and/or target type(s) of surface(s). Further, the applicator 304 can include a spray applicator, a fog applicator, a mist applicator (e.g., for a mister, a diffuser, a nebulizer, etc.), a sprinkling applicator, a spout applicator, a friction applicator (e.g., a mop head, a wipe, etc.), and/or a soaking applicator (e.g., a tub, a container, a tank, etc.), etc., among other applicators and/or types of applicators that is/are possible and contemplated herein.

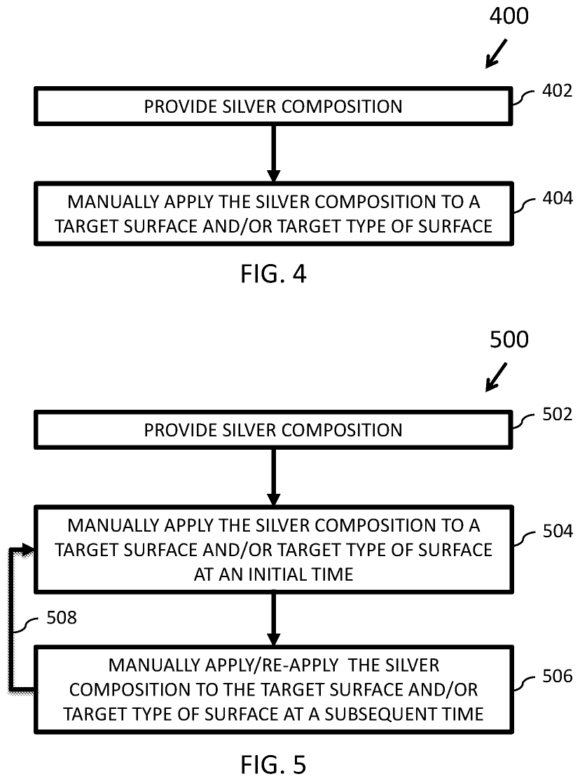

With reference to FIG. 4, FIG. 4 is a block diagram of one embodiment of a method 400 for using a silver composition to manually disinfect surfaces. At least in the illustrated embodiment, the method 400 can begin by a providing a silver composition to an applicator device 300 (block 402).

In some embodiments, the silver composition includes an amount of silver particles 100 (e.g., Ag particles), which can include an amount of silver particles 100 in the range of about 10 ppb to about 60 ppm (e.g., 10 ppm or 32 ppm), as discussed elsewhere herein. In further embodiments, the silver composition includes an amount of H₂O₂, which can include an amount of H₂O₂ in the range of about 0.1% to about 10% (e.g., 3%), as discussed elsewhere herein.

The method 400 further includes manually applying the silver composition to a target surface and/or target type of surface (block 404). In various embodiments, the silver composition is manually applied to the target surface and/or target type of surface using an applicator device 300, which is a manually operated applicator device 300.

The target surface and/or target type of surface may include any suitable surface and/or type of surface that can harbor one or more pathogens, would benefit from the removal of one or more pathogens, and/or would benefit from maintaining a pathogen-free surface and/or type of surface. Examples of target surfaces and/or target types of surfaces can include, but are not limited to, a hard surface (e.g., a stone surface, a concrete surface, a pavement surface, a hard wood surface, a diamond surface, a metal surface, a ceramic surface, a polymer surface, a glass surface, and a hard plastic surface, etc., and combinations thereof), a firm surface (e.g., a firm wood surface, a firm plastic surface, a firm polymer surface, etc., and combinations thereof), a soft surface (e.g., a soft wood surface, a soft/flexible plastic surface, a soft/flexible polymer surface, and a textile (e.g., a natural material and/or synthetic material), etc., and combinations thereof), a natural agricultural product (e.g., a flower (e.g., petal, stem, etc.), a tree (e.g., wood, bark, leaves, etc.), an orchard, a planter/grow box, a greenhouse, a hydroponic system, a bush, a plant and/or plant product, a fruit and/or fruit product, a vegetable and/or vegetable product, soil or dirt, fertilizer, meat and/or meat product, a seed, a nut, grass (e.g., a lawn, golf course (e.g., fairway, rough, putting green, etc.), and a park, etc.), and combinations thereof), and an artificial/synthetic agricultural product (e.g., artificial turf/grass, an artificial flower, an artificial tree, an artificial bush, an artificial plant and/or plant product, an artificial fruit and/or fruit product, an artificial vegetable and/or vegetable product, an artificial meat and/or meat product, an artificial seed, an artificial nut, and combinations thereof), and combinations thereof, among other surfaces and/or types of surfaces that are possible and contemplated herein. Other non-limiting examples target surfaces and/or target types of surfaces can include, but are not limited to, an algae, a vehicle (e.g., an automobile, an aircraft, a train, a watercraft, etc.), a room (e.g., a locker room, a gym, a yoga studio, a hospital room, an operating room, a sterile/clean room, etc.) and/or the equipment used therein, stadium seating, cold sterilization, a kitchen/food preparation station, and an oil well, etc., among other surfaces and/or types of surfaces that are possible and contemplated herein.

Referring to FIG. 5, FIG. 5 is a block diagram of another embodiment of a method 500 for using a silver composition to manually disinfect surfaces. At least in the illustrated embodiment, the method 500 can begin by a providing a silver composition to an applicator device 300 (block 502).

In some embodiments, the silver composition includes an amount of silver particles 100 (e.g., Ag particles), which can include an amount of silver particles 100 in the range of about 10 ppb to about 60 ppm (e.g., 10 ppm or 32 ppm), as discussed elsewhere herein. In further embodiments, the silver composition includes an amount of H₂O₂, which can include an amount of H₂O₂ in the range of about 0.1% to about 10% (e.g., 3%), as discussed elsewhere herein.

The method 500 further includes manually applying the silver composition to a target surface and/or target type of surface at a first time (block 504). In various embodiments, the silver composition is manually applied to the target surface and/or target type of surface using an applicator device 300, which is a manually operated applicator device 300.

The target surface and/or target type of surface may include any suitable surface and/or type of surface that can harbor one or more pathogens, would benefit from the removal of one or more pathogens, and/or would benefit from maintaining a pathogen-free surface and/or type of surface. Examples of target surfaces and/or target types of surfaces can include, but are not limited to, a hard surface (e.g., a stone surface, a concrete surface, a pavement surface, a hard wood surface, a diamond surface, a metal surface, a ceramic surface, a polymer surface, a glass surface, and a hard plastic surface, etc., and combinations thereof), a firm surface (e.g., a firm wood surface, a firm plastic surface, a firm polymer surface, etc., and combinations thereof), a soft surface (e.g., a soft wood surface, a soft/flexible plastic surface, a soft/flexible polymer surface, and a textile (e.g., a natural material and/or synthetic material), etc., and combinations thereof), a natural agricultural product (e.g., a flower (e.g., petal, stem, etc.), a tree (e.g., wood, bark, leaves, etc.), an orchard, a planter/grow box, a greenhouse, a hydroponic system, a bush, a plant and/or plant product, a fruit and/or fruit product, a vegetable and/or vegetable product, soil or dirt, fertilizer, meat and/or meat product, a seed, a nut, grass (e.g., a lawn, golf course (e.g., fairway, rough, putting green, etc.), and a park, etc.), and combinations thereof), and an artificial/synthetic agricultural product (e.g., artificial turf/grass, an artificial flower, an artificial tree, an artificial bush, an artificial plant and/or plant product, an artificial fruit and/or fruit product, an artificial vegetable and/or vegetable product, an artificial meat and/or meat product, an artificial seed, an artificial nut, and combinations thereof), and combinations thereof, among other surfaces and/or types of surfaces that are possible and contemplated herein. Other non-limiting examples target surfaces and/or target types of surfaces can include, but are not limited to, an algae, a vehicle (e.g., an automobile, an aircraft, a train, a watercraft, etc.), a room (e.g., a locker room, a gym, a yoga studio, a hospital room, an operating room, a sterile/clean room, etc.) and/or the equipment used therein, stadium seating, cold sterilization, a kitchen/food preparation station, and an oil well, etc., among other surfaces and/or types of surfaces that are possible and contemplated herein.

The method 500 further includes manually applying (or re-applying) the silver composition to the target surface and/or target type of surface at a second time (e.g., a subsequent time) (block 506). In various embodiments, the silver composition is manually applied/re-applied to the target surface and/or target type of surface using the applicator device 300 and the second/subsequent time may occur no earlier than about three weeks after the initial application of the silver composition in block 504.

The method 500 further includes repeating block 506 (return 508). That is, the silver composition is manually applied/re-applied to the target surface and/or target type of surface using the applicator device 300 and each second/subsequent time may occur no earlier than about three weeks after the previous application of the silver composition in block 506.

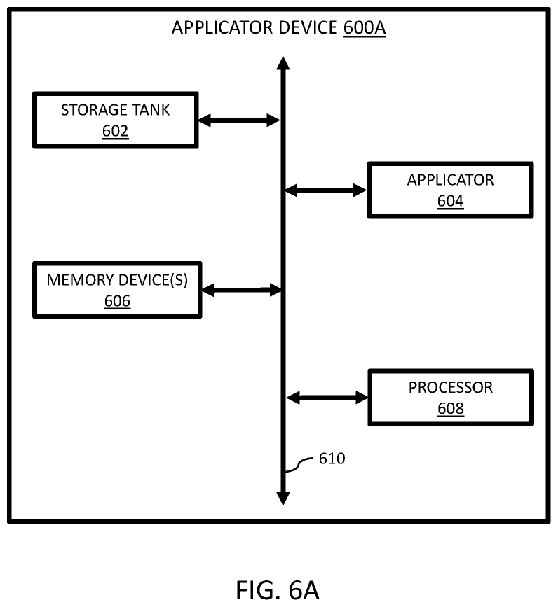

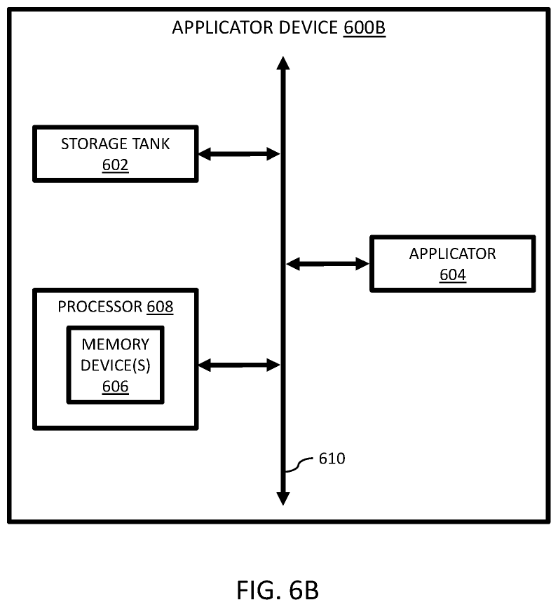

Referring to FIG. 6A, FIG. 6A is a block diagram of one embodiment of an applicator device 600A for disinfecting surfaces. The applicator device 600A may include any suitable system, apparatus, and/or device that is known or developed in the future capable of automatedly and/or automatically applying a silver composition to one or more target surfaces and/or one or more target types of surfaces and/or otherwise facilitating contact of the silver composition and the target surface(s) and/or target type(s) of surface(s).

In various embodiments, the applicator device 600A includes an automatedly and/or automatically operated applicator device 600A. Examples of an automatedly and/or automatically operated applicator device 600A can include, but are not limited to, an automatedly and/or automatically operated sprayer, an automatedly and/or automatically manually operated fogger, an automatedly and/or automatically operated mister, an automatedly and/or automatically operated sprinkler, an automatedly and/or automatically operated spout, an automatedly and/or automatically operated mop, an automatedly and/or automatically operated wiper, an automatedly and/or automatically operated diffuser, an automatedly and/or automatically operated nebulizer, and/or an automatedly and/or automatically operated soaker, etc., among other automatedly and/or automatically operated applicators that can apply a silver composition to one or more target surfaces and/or one or more target types of surfaces and/or otherwise facilitating contact of the silver composition and the target surface(s) and/or target type(s) of surface(s).

The applicator device 600A can apply a silver composition to the target surface(s) and/or target type(s) of surface(s) so that the silver composition can kill and/or destroy, on contact, one or more of the pathogens that may be present and/or harbored on the target surface(s) and/or target type(s) of surface(s). In various embodiments, the applicator device 600A can be programmed and/or configured to automatedly and/or automatically apply a silver composition at regular and/or substantially regular intervals of time to maintain a pathogen-free surface and/or type of surface. In some embodiments, the regular and/or substantially regular interval of time can include application of the silver composition every 3 weeks or 21 days, among other intervals of time that are greater than or less than 3 weeks that are possible and contemplated herein.

At least in the embodiment illustrated in FIG. 6A, the applicator device 600A includes, among other components, a storage tank 602, an applicator 604 in fluid communication with the storage tank 602, a set of memory devices 606, and a processor 608 coupled to and/or in communication with one another via a bus 610 (e.g., a wired and/or wireless bus). A storage tank 602 may include any suitable tank and/or type of tank that is known or developed in the future capable of storing a composition, sol, liquid, solution, fluid, and/or liquid including a plurality of silver particles 100 or a mixture/combination of silver particles 100 and H₂O₂. Further, the storage tank 602 may include any suitable size and/or dimensions for storing/housing a predetermined and/or predefined amount of the composition, sol, liquid, solution, fluid, and/or liquid including a plurality of silver particles 100 or a mixture/combination of silver particles 100 and H₂O₂.

An applicator 604 may include any suitable automated and/or automatic applicator and/or type of applicator that is known or developed in the future. That is, an applicator 604 may include any suitable device and/or apparatus that can apply, via automated and/or automatic operation, a composition, a sol, liquid, solution, fluid, and/or liquid including a plurality of silver particles 100 or a mixture/combination of silver particles 100 and H₂O₂ to one or more target surfaces and/or one or more target types of surfaces and/or otherwise facilitating contact of the silver composition and the target surface(s) and/or target type(s) of surface(s). Further, the applicator 604 can include a spray applicator, a fog applicator, a mist applicator (e.g., for a mister, a diffuser, a nebulizer, etc.), a sprinkling applicator, a spout applicator, a friction applicator (e.g., a mop head, a wipe, etc.), and/or a soaking applicator (e.g., a tub, a container, a tank, etc.), etc., among other automated and/or automatic applicators and/or types of applicators that is/are possible and contemplated herein.

In various embodiments, the applicator 604 is configured to turn ON/OFF in response to receiving corresponding ON/OFF commands. That is, in response to receiving an ON command, the applicator 604 is configured to turn ON and begin drawing the silver composition from in the storage tank 602 to apply the silver composition to the target surface and/or target type of surface. Conversely, in response to receiving an OFF command, the applicator 604 is configured to turn OFF and stop drawing the silver composition from in the storage tank 602 to cease applying the silver composition to the target surface and/or target type of surface.

A set of memory devices 606 may include any suitable quantity of memory devices 606. Further, a memory device 606 may include any suitable type of device and/or system that is known or developed in the future that can store computer-useable and/or computer-readable code. In various embodiments, a memory device 606 may include one or more non-transitory computer-usable mediums (e.g., readable, writable, etc.), which may include any non-transitory and/or persistent apparatus or device that can contain, store, communicate, propagate, and/or transport instructions, data, computer programs, software, code, routines, etc., for processing by or in connection with a computer processing device (e.g., processor 608).

A memory device 606, in some embodiments, includes volatile computer storage media. For example, a memory device 606 may include random access memory (RAM), including dynamic RAM (DRAM), synchronous dynamic RAM (SDRAM), and/or static RAM (SRAM). In other embodiments, a memory device 606 includes non-volatile computer storage media. For example, a memory device 606 may include a hard disk drive, a flash memory, and/or any other suitable non-volatile computer storage device that is known or developed in the future. In various embodiments, a memory device 606 includes both volatile and non-volatile computer storage media.

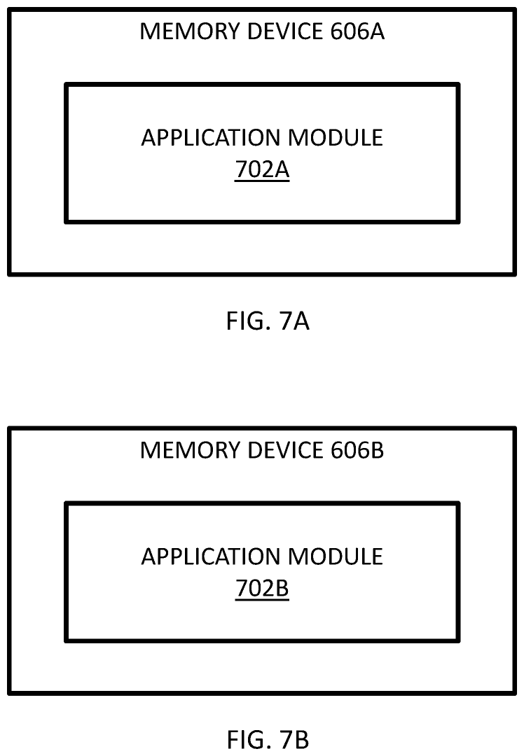

With reference now to FIG. 7A, FIG. 7A is a schematic block diagram of one embodiment of a memory device 606A. At least in the illustrated embodiment, the memory device 606A includes, among other components, an application module 702A, that is configured to operate/function when executed by the processor 608 to automatedly and/or automatically apply a silver composition to a target surface and/or target type of surface.

An application module 702A may include any suitable hardware and/or software than can apply and/or facilitate applying a silver composition to a target surface and/or target type of surface. In various embodiments, the application module 702A is configured to transmit ON/OFF commands the applicator 604.

In some embodiments, the ON command is configured to turn ON the applicator 604 so that the applicator 604 can draw the silver composition from the storage tank 602 and begin applying the silver composition to the target surface and/or target type of surface. Conversely, the OFF command is configured to turn OFF the applicator 604 so that the applicator 604 stops drawing the silver composition from in the storage tank 602 and ceases applying the silver composition to the target surface and/or target type of surface.

Referring to FIG. 7B, FIG. 7B is a schematic block diagram of another embodiment of a memory device 606B. At least in the illustrated embodiment, the memory device 606A includes, among other components, an application module 702B, that is configured to operate/function when executed by the processor 608 to automatedly and/or automatically apply a silver composition to a target surface and/or target type of surface.

An application module 702B may include any suitable hardware and/or software than can apply and/or facilitate applying a silver composition to a target surface and/or target type of surface. In various embodiments, the application module 702B is configured to transmit ON/OFF commands the applicator 604.

In some embodiments, the ON command is configured to turn ON the applicator 604 so that the applicator 604 can draw the silver composition from the storage tank 602 and begin applying the silver composition to the target surface and/or target type of surface. Conversely, the OFF command is configured to turn OFF the applicator 604 so that the applicator 604 stops drawing the silver composition from in the storage tank 602 and ceases applying the silver composition to the target surface and/or target type of surface.

In some, the application module 702B is configured to automatedly and/or automatically apply and/or facilitate applying the silver composition to the target surface and/or target type of surface at an initial time. That is, the application module 702B is configured to transmit an ON command to the applicator 604 to begin applying the silver composition to the target surface and/or target type of surface. Further, the application module 702B is configured to transmit an OFF command to the applicator 604 to cease or stop applying the silver composition to the target surface and/or target type of surface.

In some embodiments, the application module 702B is configured to transmit the OFF command to the applicator 604 in response to the expiration of a predetermined amount of time, which can include any suitable amount of time and can be based on a size of the target surface and/or target type of surface, a quantity of silver composition needed to cover the target surface and/or target type of surface, and/or an application rate of the applicator 604. The application module 702B, in additional or alternative embodiments, is configured to transmit the OFF command to the applicator 604 in response to a predetermined amount of silver composition being applied to the target surface and/or target type of surface, which can include any suitable quantity of silver composition needed to cover the target surface and/or target type of surface.

In additional embodiments, the application module 702B is configured to automatedly and/or automatically apply (or re-apply) and/or facilitate applying/re-applying the silver composition to the target surface and/or target type of surface at a second or subsequent time to the initial application of the silver composition. In various embodiments, the application module 702B is configured to automatedly and/or automatically use the applicator 604 to apply/re-apply the silver composition to the target surface and/or target type of surface and the second/subsequent time may occur no earlier than about three weeks after the initial application of the silver composition to the target surface and/or target type of surface.

Similar to above, the application module 702B is configured to transmit an ON command to the applicator 604 to begin applying the silver composition to the target surface and/or target type of surface at the subsequent time. Further, the application module 702B is configured to transmit an OFF command to the applicator 604 to cease or stop applying the silver composition to the target surface and/or target type of surface at the subsequent time.

Further, the application module 702B is configured to automatedly and/or automatically apply (or re-apply) and/or facilitate applying/re-applying the silver composition to the target surface and/or target type of surface at further subsequent times. In various embodiments, the application module 702B is configured to automatedly and/or automatically use the applicator 604 to apply/re-apply the silver composition to the target surface and/or target type of surface at each subsequent time, which may occur no earlier than about three weeks after the previous application of the silver composition to the target surface and/or target type of surface.

Similar to above, the application module 702B is configured to transmit an ON command to the applicator 604 to begin applying the silver composition to the target surface and/or target type of surface at each subsequent time. Further, the application module 702B is configured to transmit an OFF command to the applicator 604 to cease or stop applying the silver composition to the target surface and/or target type of surface at each subsequent time.

Referring back to FIG. 6A, a processor 608 may include any suitable non-volatile/persistent hardware and/or software configured to perform and/or facilitate performing various processing functions and/or operations. In various embodiments, the processor 608 includes hardware and/or software for executing instructions in the application module 702A and/or 702B. The application module 702A and/or 702B executed by the processor 608 can be stored on and executed from a memory device 606 and/or from the processor 608.

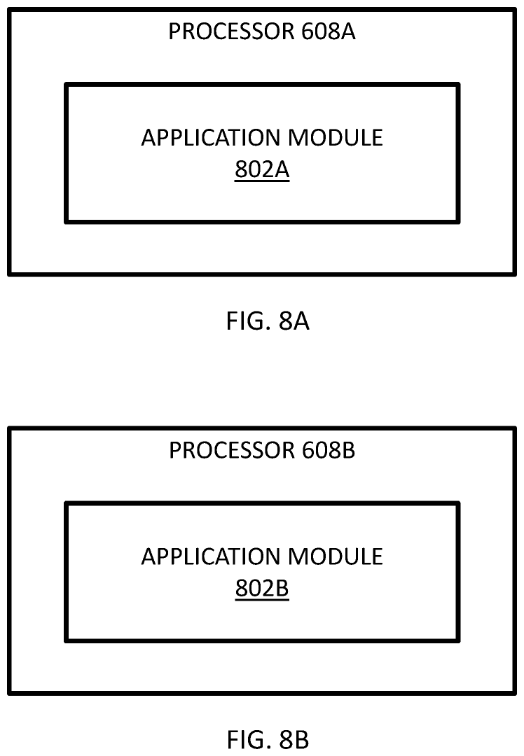

With reference to FIG. 8A, FIG. 8A is a schematic block diagram of one embodiment of a processor 608A. At least in the illustrated embodiment, the processor 608A includes, among other components, an application module 802A similar to the application module 702A in the memory device 606A discussed with reference to FIG. 7A.

Referring to FIG. 8B, FIG. 8B is a schematic block diagram of another embodiment of a processor 608B. At least in the illustrated embodiment, the processor 608B includes, among other components, an application module 802B similar to the application module 702B in the memory device 606B discussed with reference to FIG. 7B.

With reference to FIG. 6B, FIG. 6B is a block diagram of another embodiment of an applicator device 600B. The applicator device 600B includes, among other components, a storage tank 602, an applicator 604 in fluid communication with the storage tank 602, a set of memory devices 606, and a processor 608 coupled to and/or in communication with one another via a bus 610 similar to the storage tank 602, applicator 604, memory device(s) 606, processor 608, and bus 610 of the applicator device 600A discussed with reference to FIG. 6A. Alternative to the applicator device 600A, the processor 608 in the applicator device 600B includes the memory device(s) 606 therein as opposed to the memory device(s) 606 of the applicator device 600A being a different device than and/or independent of the processor 608.

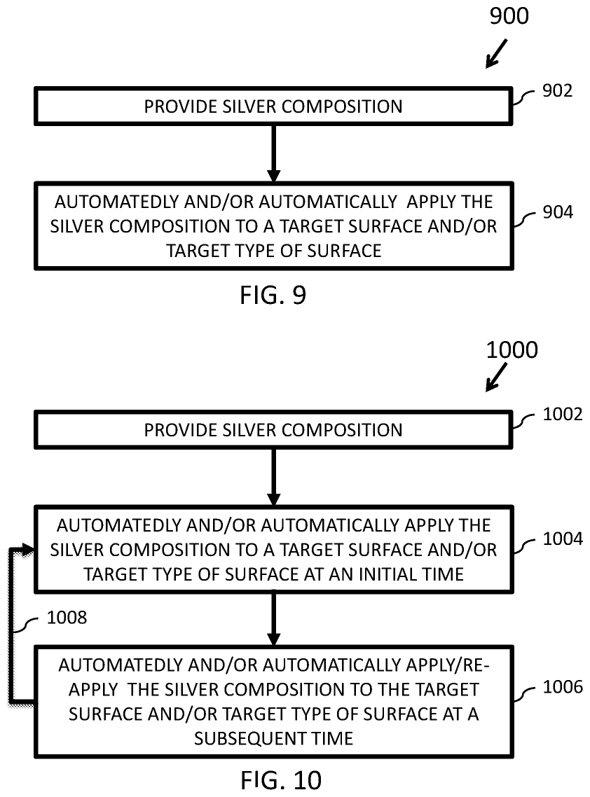

With reference to FIG. 9, FIG. 9 is a block diagram of one embodiment of a method 900 for using a silver composition to automatedly and/or automatically disinfect surfaces. At least in the illustrated embodiment, the method 900 can begin by providing a silver composition to an applicator device 600 (block 902).

In some embodiments, the silver composition includes an amount of silver particles 100 (e.g., Ag particles), which can include an amount of silver particles 100 in the range of about 10 ppb to about 60 ppm (e.g., 10 ppm or 32 ppm), as discussed elsewhere herein. In further embodiments, the silver composition includes an amount of H₂O₂, which can include an amount of H₂O₂ in the range of about 0.1% to about 10% (e.g., 3%), as discussed elsewhere herein.

The method 900 further includes a processor (e.g., processor 608A or 608B) automatedly and/or automatically applying the silver composition to a target surface and/or target type of surface (block 904). In various embodiments, the silver composition is automatedly and/or automatically applied to the target surface and/or target type of surface using an applicator device 600, which is an automatedly and/or automatically operated applicator device 600. The target surface and/or target type of surface may include any suitable surface and/or type of surface (e.g., a hard surface, a firm surface, a soft surface, and/or an agricultural surface, etc.) that can harbor one or more pathogens, would benefit from the removal of one or more pathogens, and/or would benefit from maintaining a pathogen-free surface and/or type of surface similar to the various embodiments and/or examples discussed elsewhere herein.

Referring to FIG. 10, FIG. 10 is a block diagram of another embodiment of a method 1000 for using a silver composition to automatedly and/or automatically disinfect surfaces. At least in the illustrated embodiment, the method 1000 can begin by a providing a silver composition to an applicator device 600 (block 1002).

In some embodiments, the silver composition includes an amount of silver particles 100 (e.g., Ag particles), which can include an amount of silver particles 100 in the range of about 10 ppb to about 60 ppm (e.g., 10 ppm or 32 ppm), as discussed elsewhere herein. In further embodiments, the silver composition includes an amount of H₂O₂, which can include an amount of H₂O₂ in the range of about 0.1% to about 10% (e.g., 3%), as discussed elsewhere herein.

The method 1000 further includes a processor (e.g., processor 608B) automatedly and/or automatically applying the silver composition to a target surface and/or target type of surface at a first time (block 1004). In various embodiments, the silver composition is automatedly and/or automatically applied to the target surface and/or target type of surface using an applicator device 600, which is an automatedly and/or automatically operated applicator device 600. The target surface and/or target type of surface may include any suitable surface and/or type of surface (e.g., a hard surface, a firm surface, a soft surface, and/or an agricultural surface, etc.) that can harbor one or more pathogens, would benefit from the removal of one or more pathogens, and/or would benefit from maintaining a pathogen-free surface and/or type of surface similar to the various embodiments and/or examples discussed elsewhere herein.

The method 1000 further includes the processor 608B automatedly and/or automatically applying (or re-applying) the silver composition to the target surface and/or target type of surface at a second time (e.g., a subsequent time) (block 1006). In various embodiments, the silver composition is automatedly and/or automatically applied/re-applied to the target surface and/or target type of surface using the applicator device 600 and the second/subsequent time may occur no earlier than about three weeks after the initial application of the silver composition in block 1004.

The method 1000 further includes the processor 608B automatedly and/or automatically repeating block 1006 at least one additional time (return 1008). That is, the silver composition is automatedly and/or automatically applied/re-applied to the target surface and/or target type of surface using the applicator device 600 and each second/subsequent time may occur no earlier than about three weeks after the previous application of the silver composition in block 1006.

One unexpected exemplary use of a silver composition disclosed herein includes termite control. In some embodiments, the silver composition is applied to a target wood surface and/or other target surface in which the silver composition can be ingested by a termite. After a termite ingests the silver composition, the silver composition kills the bacteria in the termite's gut used to break down the wood and/or wood pulp, which prevents the termite from obtaining the nutrients from the wood and/or wood pulp (e.g., a termite's food). Because the termite can no longer obtain the nutrients from its food, the termite will starve to death even though its stomach is full of food. In this manner, the silver compositions disclosed herein can eliminate/kill termites in an unexpected manner that is chemical-free and/or safe for the environment (e.g., environmentally friendly). That is, it was unexpected that a silver composition that is capable of disinfecting a target surface could also result in controlling and/or killing a population of termites.

While specific efficacy time periods are disclosed herein, the various embodiments are not limited to these specific efficacy time periods. That is, various embodiments may include greater efficacy time periods or smaller efficacy time periods than the specified efficacy time periods.

Similarly, while specific time periods between applications of a silver compound are disclosed herein, the various embodiments are not limited to these specific time periods between applications. That is, various embodiments may include greater time periods between applications or smaller time periods between applications than the specified time periods between applications. For example, some time periods between applications can depend on and/or be based on any suitable factor(s) including, but not limited to, for example, the silver composition, the type of silver composition, the surface, the type of surface, the use of a surface, the type of use of a surface, an environment surrounding a surface or type of surface, and/or a type of environment surrounding a surface or type of surface, etc., among other factors that are possible and contemplated herein.

Aspects of the various embodiments discussed above may be embodied as a method, apparatus, or program product for using silver compositions to manually or automatedly and/or automatically disinfect surfaces. Accordingly, embodiments may take the form of an entirely hardware embodiment, an entirely software embodiment (including firmware, resident software, micro-code, etc.) or an embodiment combining software and hardware aspects that may all generally be referred to herein as a circuit, module, or system. Furthermore, embodiments may take the form of a program product embodied in one or more computer-readable storage devices storing machine readable code, computer-readable code, and/or program code, referred hereafter as code. The storage devices may be tangible, non-transitory, and/or non-transmission. The storage devices may not embody signals. In a certain embodiment, the storage devices only employ signals for accessing code.

Certain of the functional units described in this specification have been labeled as modules to more particularly emphasize their implementation independence. For example, a module may be implemented as a hardware circuit comprising custom very-large-scale integration (VLSI) circuits or gate arrays, off-the-shelf semiconductors such as logic chips, transistors, or other discrete components. A module may also be implemented in programmable hardware devices such as field programmable gate arrays, programmable array logic, programmable logic devices or the like.

Modules may also be implemented in code and/or software for execution by various types of processors. An identified module of code may, for instance, include one or more physical or logical blocks of executable code which may, for instance, be organized as an object, procedure, or function. Nevertheless, the executables of an identified module need not be physically located together and may include disparate instructions stored in different locations which, when joined logically together, include the module and achieve the stated purpose for the module.

Indeed, a module of code may be a single instruction, or many instructions, and may even be distributed over several different code segments, among different programs, and across several memory devices. Similarly, operational data may be identified and illustrated herein within modules and may be embodied in any suitable form and organized within any suitable type of data structure. The operational data may be collected as a single data set or may be distributed over different locations including over different computer-readable storage devices. Where a module or portions of a module are implemented in software, the software portions are stored on one or more computer-readable storage devices.

Any combination of one or more computer-readable media may be utilized. The computer-readable medium/media may include one or more computer-readable storage media. The computer-readable storage medium/media may be a storage device storing the code. The storage device may be, for example, but not limited to, an electronic, magnetic, optical, electromagnetic, infrared, holographic, micromechanical, or semiconductor system, apparatus, or device, or any suitable combination of the foregoing.

More specific examples (e.g., a non-exhaustive and/or non-limiting list) of the storage device would include the following: an electrical connection having one or more wires, a portable computer diskette, a hard disk, a random-access memory (RAM), a read-only memory (ROM), an erasable programmable read-only memory (EPROM or Flash memory), a portable compact disc read-only memory (CD-ROM), an optical storage device, a magnetic storage device, or any suitable combination of the foregoing. In the context of this document, a computer-readable storage medium may be any tangible medium that can contain or store a program for use by or in connection with an instruction execution system, apparatus, or device.

Code for carrying out operations for embodiments may be written in any combination of one or more programming languages including an object-oriented programming language such as Python, Ruby, Java, Smalltalk, C++, or the like, and conventional procedural programming languages, such as the C programming language, or the like, and/or machine languages such as assembly languages. The code may execute entirely on the user's computer, partly on the user's computer, as a stand-alone software package, partly on the user's computer and partly on a remote computer or entirely on the remote computer or server. In the latter scenario, the remote computer may be connected to the user's computer through any type of network, including a local area network (LAN) or a wide area network (WAN), or the connection may be made to an external computer (for example, through the Internet using an Internet Service Provider).

Reference throughout this specification to one embodiment, an embodiment, or similar language means that a particular feature, structure, or characteristic described in connection with the embodiment is included in at least one embodiment. Thus, appearances of the phrases in one embodiment, in an embodiment, and similar language throughout this specification may, but do not necessarily, all refer to the same embodiment, but mean one or more but not all embodiments unless expressly specified otherwise. The terms including, comprising, having, and variations thereof mean including but not limited to, unless expressly specified otherwise. An enumerated listing of items does not imply that any or all of the items are mutually exclusive, unless expressly specified otherwise. The terms, “a,” “an,” and “the,” also refer to one or more unless expressly specified otherwise.

In addition, as used herein, the term, “set,” can mean one or more, unless expressly specified otherwise. The term, “sets,” can mean multiples of or a plurality of one or mores, ones or more, and/or ones or mores consistent with set theory, unless expressly specified otherwise.

Furthermore, the described features, structures, or characteristics of the embodiments may be combined in any suitable manner. In the above description, numerous specific details are provided, such as examples of programming, software modules, user selections, network transactions, database queries, database structures, hardware modules, hardware circuits, hardware chips, etc., to provide a thorough understanding of embodiments. One skilled in the relevant art will recognize, however, that embodiments may be practiced without one or more of the specific details, or with other methods, components, materials, and so forth. In other instances, well-known structures, materials, or operations are not shown or described in detail to avoid obscuring aspects of an embodiment.

Aspects of the embodiments are described above with reference to schematic flowchart diagrams and/or schematic block diagrams of methods, apparatus, systems, and program products according to embodiments. It will be understood that at least some blocks of the schematic flowchart diagrams and/or schematic block diagrams, and combinations of blocks in the schematic flowchart diagrams and/or schematic block diagrams, can be implemented by code. The code may be provided to a processor of a general-purpose computer, special-purpose computer, or other programmable data processing apparatus to produce a machine, such that the instructions, which execute via the processor of the computer or other programmable data processing apparatus, create means for implementing the functions/acts specified in the schematic flowchart diagrams and/or schematic block diagrams block or blocks.

The code may also be stored in a storage device that can direct a computer, other programmable data processing apparatus, or other devices to function in a particular manner, such that the instructions stored in the storage device produce an article of manufacture including instructions which implement the function/act specified in the schematic flowchart diagrams and/or schematic block diagrams block or blocks.

The code may also be loaded onto a computer, other programmable data processing apparatus, or other devices to cause a series of operational steps to be performed on the computer, other programmable apparatus or other devices to produce a computer implemented process such that the code which execute on the computer or other programmable apparatus provide processes for implementing the functions/acts specified in the flowchart and/or block diagram block or blocks.

The schematic flowchart diagrams and/or schematic block diagrams in the Figures illustrate the architecture, functionality, and operation of possible implementations of apparatuses, systems, methods and program products according to various embodiments. In this regard, each block in the schematic flowchart diagrams and/or schematic block diagrams may represent a module, segment, or portion of code, which includes one or more executable instructions of the code for implementing the specified logical function(s).

It should also be noted that, in some alternative implementations, the functions noted in the block may occur out of the order noted in the Figures. For example, two blocks shown in succession may, in fact, be executed substantially concurrently, or the blocks may sometimes be executed in the reverse order, depending upon the functionality involved. Other steps and methods may be conceived that are equivalent in function, logic, or effect to one or more blocks, or portions thereof, of the illustrated Figures.

Although various arrow types and line types may be employed in the flowchart and/or block diagrams, they are understood not to limit the scope of the corresponding embodiments. Indeed, some arrows or other connectors may be used to indicate only the logical flow of the depicted embodiment. For instance, an arrow may indicate a waiting or monitoring period of unspecified duration between enumerated steps of the depicted embodiment. It will also be noted that each block of the block diagrams and/or flowchart diagrams, and combinations of blocks in the block diagrams and/or flowchart diagrams, can be implemented by special purpose hardware-based systems that perform the specified functions or acts, or combinations of special purpose hardware and code.

The description of elements in each figure may refer to elements of proceeding figures. Like numbers refer to like elements in all figures, including alternate embodiments of like elements.

The various disclosed embodiments may be practiced in other specific forms. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A method, comprising:
providing a composition to an applicator device, the composition comprising hydrogen peroxide (H₂O₂) and silver (Ag) particles; and
applying the composition to a target type of surface.

2. The method of claim 1, wherein the composition comprises 0.1% to 5% H₂O₂.

3. The method of claim 2, wherein the composition comprises 10 ppb to 60 ppm of the Ag particles.

4. The method of claim 1, wherein the composition comprises 3% H₂O₂ and 10 ppm of the Ag particles.

5. The method of claim 4, wherein the Ag particles comprise one of nano-silver particles, colloidal silver particles, and ionic silver particles.

6. The method of claim 1, wherein:
the applicator device comprises a manually operated applicator device;
the manually operated applicator device comprises one of a manually operated sprayer, a manually operated fogger, a manually operated mister, a manually operated sprinkler, a manually operated spout, a manually operated mop, a manually operated wiper, a manually operated diffuser, a manually operated nebulizer, and a manually operated soaker; and
applying the composition comprises manually applying the composition via the manually operated applicator device.

7. The method of claim 1, wherein:
the applicator device includes an automated applicator device;
the automated applicator device comprises one of an automated sprayer, an automated fogger, an automated mister, an automated sprinkler, an automated spout, an automated mop, an automated wiper, an automated diffuser, an automated nebulizer, and an automated soaker; and
applying the composition comprises automatedly applying the composition via the automated applicator device.

8. The method of claim 1, wherein the target type of surface comprises one of a hard surface, a firm surface, a soft surface, and an agricultural surface.

9. The method of claim 8, wherein applying the composition to the target type of surface comprises applying the composition to the one of the hard surface, the firm surface, the soft surface, and the agricultural surface at a first time and at one or more second times that are not less than twenty-one days after a previous application of the composition to the one of the hard surface, the firm surface, the soft surface, and the agricultural surface.

10. The method of claim 1, wherein applying the composition to the target type of surface comprises applying the composition to the target type of surface at a first time and at one or more second times that are not less than twenty-one days after a previous application of the composition to the target type of surface.

11. An apparatus, comprising:
an applicator device configured to store a composition comprising hydrogen peroxide (H₂O₂) and silver (Ag) particles;
a processor; and
a memory configured to store code executable by the processor to automatedly apply, via the applicator device, the composition to a target type of surface.

12. The apparatus of claim 11, wherein the composition comprises 0.1% to 5% H₂O₂.

13. The apparatus of claim 12, wherein the composition comprises 10 ppb to 60 ppm of the Ag particles.

14. The apparatus of claim 11, wherein the composition comprises 3% H₂O₂ and 10 ppm of the Ag particles.

15. The apparatus of claim 14, wherein the Ag particles comprise one of nano-silver particles, colloidal silver particles, and ionic silver particles.

16. The apparatus of claim 11, wherein:
the applicator device includes an automated applicator device;
the automated applicator device comprises one of an automated sprayer, an automated fogger, an automated mister, an automated sprinkler, an automated spout, an automated mop, an automated wiper, an automated diffuser, an automated nebulizer, and an automated soaker; and
automatedly applying the composition comprises automatedly applying the composition via the automated applicator device.

17. The apparatus of claim 11, wherein the target type of surface comprises one of a hard surface, a firm surface, a soft surface, and an agricultural surface.

18. The apparatus of claim 17, wherein automatedly applying the composition to the target type of surface comprises automatedly applying the composition to the one of the hard surface, the firm surface, the soft surface, and the agricultural surface at a first time and at one or more second times that are not less than twenty-one days after a previous application of the composition to the one of the hard surface, the firm surface, the soft surface, and the agricultural surface.

19. The apparatus of claim 11, wherein applying the composition to the target type of surface comprises applying the composition to the target type of surface at a first time and at one or more second times that are not less than twenty-one days after a previous application of the composition to the target type of surface.

20. A computer program product comprising a computer-readable storage device including code embodied therewith, the code executable by a processor to cause the processor to:
automatedly facilitate providing a composition comprising hydrogen peroxide and silver particles to an applicator device; and
automatedly facilitate applying, via the applicator device, the composition to a target type of surface.
